(19) United States
(12) Patent Application Publication
(10) Pub. No.: US 2025/0053739 A1
(43) Pub. Date: Feb. 13, 2025

(54) A method and system for predicting human fall risk based on electronic nursing text data

(71) Applicant: CHONGQING UNIVERSITY OF POSTS AND TELECOMMUNICATIONS, Chongqing (CN)

(72) Inventors: Haiyan Yu, Chongqing (CN); Xiaolong Zuo, Chongqing (CN); Yi Yan, Chongqing (CN); Guokang Fan, Chongqing (CN)

(73) Assignee: CHONGQING UNIVERSITY OF POSTS AND TELECOMMUNICATIONS, Chongqing (CN)

(21) Appl. No.: 18/272,584

(22) PCT Filed: Oct. 24, 2022
(86) PCT No.: PCT/CN2022/126882

§ 371 Date: Oct. 20, 2024

(30) Foreign Application Priority Data
Apr. 19, 2022 (CN) CN 202210409172.9

(51) Int. Cl. G06F 40/279; G16H 50/20; G16H 50/70

(52) U.S. Cl. CPC G06F 40/279; G16H 50/20; G16H 50/70

(57) ABSTRACT

The present invention includes techniques for data processing and particularly relates to a method and system for predicting human fall risk based on electronic nursing text (ENT) data. The method comprises the following steps: obtaining an ENT data set, pre-processing data in the ENT data set, and constructing a Morse Fall Scale (MFS) dictionary with the pre-processed data; extracting features from the ENT data of patients to be predicted with a natural language processing technology; analyzing the extracted text features by the MFS dictionary to obtain a data set of risk factors; training a decision tree algorithm by using the data set of risk factors to obtain a prediction result of the patient's fall risk; clustering and precisely nursing the patients according to the prediction result. The invention constructs the MFS dictionary through the electronic health records to obtain fall risk factors of the patients, and iteratively predicts their fall risks according to the risk factors, thereby improving the prediction efficiency.

### TECHNICAL FIELD

The present invention includes techniques for data processing and particularly relates to a method and system for predicting human fall risk based on electronic nursing text data.

### BACKGROUND

Fall risk factors include those associated with the person receiving nursing, organizational or environmental factors, and behavioral activities at the time of the fall. The factor assessment of fall risk is only a small part of fall prevention. In busy and understaffed nursing centers, distilling well-established guidelines for avoiding falls is a challenge that it must strike a balance between the movement freedom of people and the risk of serious injury. A nursing text dialogue mechanism provides the nursing team, as well as the nursing recipient and family, with decision support information about the patient, including fall risk factors, advice on prevention strategies, and coping strategies after a fall. Based on the nursing text data from the electronic health records (EHR), computable ontology knowledge is developed to characterize the existing knowledge of fall risk management and set corresponding nursing plans for elderly people with different levels of risk, thereby optimizing the nursing plan.

Comparing manual annotation with automatic annotation based on Morse Fall Scale (MFS) dictionary, although both have higher accuracy, manual annotation is much less efficient than automatic annotation that it requires repeated checks during the annotation process and has a higher probability of errors and omissions.

### INVENTION CONTENT

The present invention provides a method for predicting human fall risk based on electronic nursing text (ENT) data, which comprises: obtaining an ENT data set, pre-processing data in the ENT data set, and constructing a Morse Fall Scale (MFS) dictionary with the pre-processed data; extracting features from the ENT data of patients to be predicted with a natural language processing technology; analyzing the extracted text features by the MFS dictionary to obtain a data set of risk factors; training a decision tree algorithm by using the data set of risk factors to obtain a prediction result of the patient's fall risk; clustering and precisely nursing the patients according to the prediction result.

Preferably, the process of constructing the MFS dictionary comprises: performing sentiment score mining and fall dictionary score mining on all ENT data in the ENT data set; constructing the MFS dictionary based on the results of sentiment score mining and fall dictionary score mining.

Further, the process of performing sentiment score mining on the ENT data comprises: using Jieba word segmentation tool to the ENT data to obtain vector phrases; using natural language processing technology to extract sentiment words of the vector phrases; classifying all sentiment words into sentiment words with negation, sentiment words without negation, and other sentiment words; respectively applying sentiment word score mechanisms of sentiment words with negation, sentiment words without negation, and other sentiment words to compute sentiment scores of sentiment words with negation, sentiment words without negation, and other sentiment words; summing all the sentiment scores to get total score for the sentiment words.

Further, the process of applying sentiment word score mechanism of sentiment words with negation to calculate sentiment score of sentiment words with negation includes:
Step 1: segmenting the document into words to find the sentiment words, negation words and adverbs of degree;
Step 2: determining whether each sentiment word is preceded by negation word and adverb of degree, and combining the negation word and the adverb of degree into a group;
Step 3: computing score of the sentiment words with negation and weight of the adverb of degree according to the NLP dictionary; multiplying sentiment weight of the sentiment words by −1 if there is a negation; multiplying degree value of the adverb of degree if there is an adverb of degree;
Step 4: taking opposite number of initial score and then multiplying it with the weight of the adverb of degree to get the sentiment score of the sentiment words with negations; summing up the sentiment scores of sentiment words; those greater than 0 are classified as positive, and those less than 0 are classified as negative, wherein the absolute magnitude of the sentiment score reflects the degree of positivity or negativity.

Further, the weight of the adverb of degree is calculated by formula:

$$Score ( w ) = {log}_{2} \frac{freq ( w , positive ) * freq ( negative )}{freq ( w , negative ) * freq ( positive )}$$

wherein, freq (w, positive) is the number of occurrences of a word w in positive texts, freq (positive) is the total number of each word in each nursing text, freq (negative) is the total number of negative words in each nursing text, and freq (w, negative) is the number of occurrences of a word w in negative text.

Preferably, the process of applying sentiment word score mechanism of sentiment words without negation to calculate sentiment score of sentiment words without negation includes: calculating initial score of sentiment words without negation and degree adverb weight; multiplying the initial score with the degree adverb weight to obtain the sentiment score of the sentiment words without negation.

Preferably, the process of performing fall dictionary score mining on the ENT data includes: constructing fall dictionary; using Jieba word segmentation tool to the ENT data to obtain vector phrases; using the fall dictionary to extract fall words in the vector phrases; calculating score of each fall word, and summing all the scores to obtain fall dictionary score.

Preferably, data in the data set of risk factors includes fall level, fall history, secondary diagnosis result, crutch, cane, walker, intravenous appliances/heparin lock or saline indicators, gait/mobility, mental status, sentiment score and the Morse Fall Scale.

Preferably, the process of processing the data in the data set of risk factors using the decision tree algorithm comprises:
Step 1: constructing decision tree, taking Morse Fall Scale in the data set of risk factors as root node of the decision tree, and classifying patients according to the root node;
Step 2: querying each subclass to determine whether the classification result of each subclass is correct; if yes, taking branch end node as leaf node of the decision tree; otherwise, selecting a non-parent node attribute and repeating the Step 1;
Step 3: selecting a non-parent node's attribute, and continuing to classify results in the Step 1 according to attribute score; classification result is final prediction result.

A system for predicting human fall risk based on ENT data, which comprises: a data acquisition module, a data pre-processing module, a text feature extraction module, a MFS dictionary module, an iterative risk prediction module, a fall event prevention and control module, and a feedback module;
the data acquisition module is used for acquiring the user's ENT data and inputting the data into the data pre-processing module;
the data pre-processing module is used for pre-processing the ENT data, wherein the pre-processing comprises filtering the ENT data for corresponding features, removing duplicate features, and completing missing features;
the text feature extraction module is used for extracting features from the ENT data processed by the data pre-processing module;
the MFS dictionary module is used for analyzing the extracted text features to obtain the data set of risk factors;
the iterative risk prediction module is used for selecting features in the data set of risk factors using a decision tree algorithm to obtain prediction result of patient's fall risk, and inputting the prediction result into the fall event prevention and control module;
the fall event prevention and control module is used for constructing fall risk prevention strategy based on the prediction result;
the feedback module is used to feedback the fall risk prevention strategy generated by the fall event prevention and control module to the user.

The benefits of the present invention are that the invention constructs the MFS dictionary through the electronic health records to obtain fall risk factors of the patients, and iteratively predicts their fall risks according to the risk factors, thereby improving the prediction efficiency. Also, intelligent decision-making support in the present invention effectively saves labor costs and avoids human errors.

Other advantages, objectives and features of the present invention will be illustrated in the following description and will be apparent to those skilled in the art based on the following investigation or can be taught from the practice of the present invention.

### DESCRIPTION OF DRAWINGS

To enable the purpose, the technical solution and the advantages of the present invention to be more clear, the present invention will be preferably described in detail below in combination with the drawings, wherein:

FIG. 1 is a flowchart of the human fall risk prediction of the present invention;

FIG. 2 is a flowchart of the sentiment score mining of the present invention;

FIG. 3 is a flowchart of the fall dictionary score mining of the present invention;

FIG. 4 is a flowchart for extracting the data set of the present invention;

FIG. 5 is a flowchart of the data processing by the decision tree algorithm of the present invention;

FIG. 6 is a pedigree diagram of the present invention;

FIG. 7 shows a human fall risk prediction system based on electronic nursing text (ENT) data.

### DETAILED DESCRIPTION

Embodiments of the present invention are described as follows. Those skilled in the art can understand the related advantages and effects of the present invention through the disclosure of the description. The present invention can also be implemented or applied with additional specific embodiments. All details in the description can be modified or adapted based on different perspectives and applications without departing from the essential content of the present invention. It should be noted that the figures provided in the following embodiments only exemplarily explain the basic conception of the present invention, and if there is no conflict, the following embodiments and their features can be mutually combined.

The present invention provides a method for human fall risk prediction based on electronic nursing text (ENT) data, which includes: firstly, pre-processing the acquired ENT data set to obtain risk prediction requirements related to fall events and define cases; secondly, constructing an ontology engine includes a fall domain ontology knowledge base to ensure that the decision-making support system for electronic nursing and the electronic nursing profile system is adaptive and operational. According to health informatics standards, the mapping service will use ontology knowledge and a well-known nursing terminology system for mapping. Again, a machine learning and inference engine is constructed to complete the context-adaptive decision-making tree model and systematic clustering algorithm for fall risk knowledge extraction which includes fall risk factor extraction, potential response of fall prevention and evidence chain management. Fourth, a fall event-related control panel is constructed for patients to use, and its effectiveness is verified by a demonstration application with a decision-making support control panel. This control panel is embeddedly integrated with the existing EHR system to provide the nursing team as well as the nursing recipient and family with decision-making support information about the person through a nursing text dialogue mechanism, including fall risk factors, recommendations related to prevention strategies, after-fall response strategies and the user experience of the system.

The present invention will collect relevant records of nursing staff caring for the elderly, analyze the risk degree of fall of the elderly through text mining method of the fall dictionary score and the sentiment score, and extract the human body (patient, etc.) fall risk factor, thereby realizing the fall risk prediction based on the ENT data. First, the Morse Fall Scale (MFS) and Natural Language Processing (NLP) base are extended to knowledge base toolkit to parse unstructured nursing text data. Second, the parsed data are mapped to ontology knowledge, i.e., to well-known nursing terminology systems including ICD-11, Morse Fall Scale System, Minimum Nursing Set NMDF, and National Health Council WS45.7-2004, which are explicit, formal, and shareable specifications of the conceptual system related to fall events. According to health informatics standards, the mapping service uses ontology knowledge and minimum nursing data set to extract the data set of risk factors. Again, the individual nursing texts are used as case sets, and their values are mapped to the variable set to obtain a decision-making data set for each case. This decision-making data set includes both attribute variables and decision-making variables. The attribute variables are derived from the characteristics of the cases, while the decision-making variables are given by the Morse Fall Scale for each case data. Finally, the decision-making dataset is trained by a decision-making tree model which enables the fall event prediction for new cases. The invention is capable of predicting the potential response to a fall event and managing the evidence chain and predicting fall risk through cases and a knowledge base.

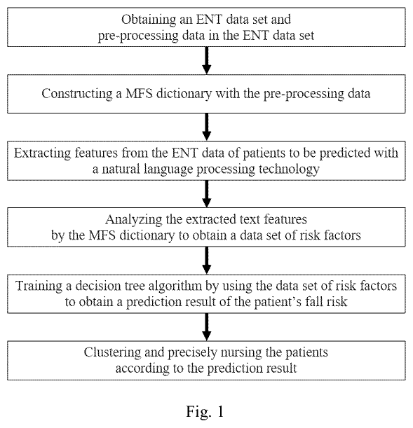

A method for predicting human fall risk based on ENT data, as shown in FIG. 1, which comprises: obtaining an ENT data set, pre-processing data in the ENT data set, and constructing a Morse Fall Scale (MFS) dictionary with the pre-processed data; extracting features from the ENT data of patients to be predicted with a natural language processing technology; analyzing the extracted text features by the MFS dictionary to obtain a data set of risk factors; training a decision tree algorithm by using the data set of risk factors to obtain a prediction result of the patient's fall risk; clustering and precisely nursing the patients according to the prediction result. A specific embodiment of a human fall risk prediction method based on electronic care text data, wherein the method includes: extending the Morse Fall Scale score and a natural language processing (NLP) library into a knowledge base toolkit to parse unstructured care text data; mapping parsed data to relevant data variables and values defined in the ontology knowledge. This provides automated processing of text data (nursing progress report) conditions to extract fall risk factors, prevent potential responses, manage fall risk evidence chain and propose countermeasures.

The process of constructing the MFS dictionary comprises: obtaining ENT data of different patients; performing sentiment score mining and fall dictionary score mining on all ENT data; constructing the MFS dictionary based on the results of sentiment score mining and fall dictionary score mining.

The process of performing sentiment score mining on the ENT data comprises: using Jieba word segmentation tool to the ENT data to obtain vector phrases; using natural language processing technology to extract sentiment words of the vector phrases; classifying all sentiment words into sentiment words with negation, sentiment words without negation, and other sentiment words; respectively applying sentiment word score mechanisms of sentiment words with negation, sentiment words without negation, and other sentiment words to calculate sentiment scores of sentiment words with negation, sentiment words without negation, and other sentiment words; summing all the sentiment scores to get total score for the sentiment words.

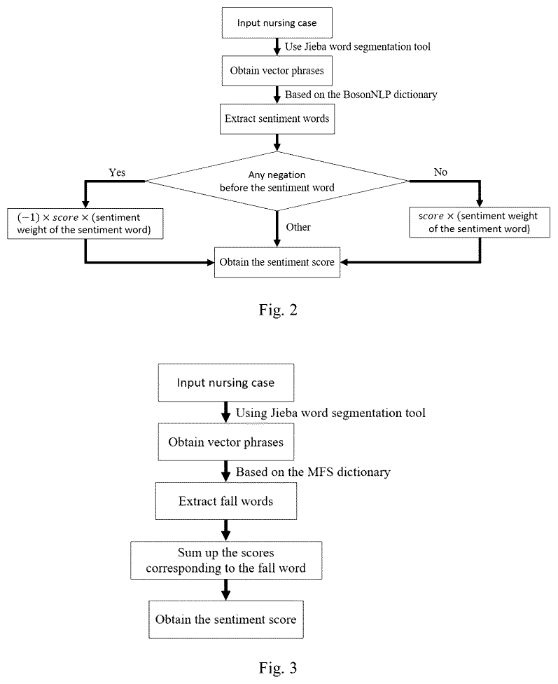

Sentiment tendency of electronic nursing file is a tendency of the scoring subject (nurse, etc.) to the test object (e.g., the elderly) through the electronic nursing file to obtain the subjective inner likes and dislikes and inner evaluations. The attitude of the elderly towards their physical condition is a key factor in the occurrence of fall. Different attitudes determine the probability of fall to a certain extent. Therefore, the sentiment score mining is used to score the sentiment of the elderly in each case, and provide different emotional guidance according to their scores, so that the elderly can have a positive and optimistic state of mind about their physical condition and life status, thus reducing the risk of falls. As shown in FIG. 2, the process of performing sentiment score mining on the ENT data comprises:
Step 1: importing patient life;
Step 2: using Jieba word segmentation tool to obtain vector phrases; Jieba word segmentation tool is a widely used, effective and open source word segmentation tool; based on prefix dictionaries to achieve efficient word graph scanning, a directed acyclic graph (DAG) of all possible word formation cases in a sentence is generated, which uses dynamic planning to find the path with maximum probability to find the maximum cut combination based on word frequency; for unregistered words, the word formation ability based on the HMM model (Hidden Markov Model) is used, using the Viterbi algorithm. Jieba supports customized professional dictionaries and unregistered dictionaries;
Step 3: obtaining sentiment words based on BosonNLP dictionary;

Step 4: iterating through the obtained sentiment words;

Step 5: the score is the sum of the adverb weight multiplied by the score for adverbs of degree only and the adverb weight multiplied by the opposite of the score for both adverbs of degree and negation;
Step 6: when the score is greater than 0, the higher score means that the patient has a more positive state of mind; when the score is less than 0, the lower score means that the patient has a more negative state of mind.

As shown in FIG. 3, the process of performing fall dictionary score mining on the ENT data includes: constructing fall dictionary; using Jieba word segmentation tool to the ENT data to obtain vector phrases; using the fall dictionary to extract fall words in the vector phrases; calculating score of each fall word, and summing all the scores to obtain fall dictionary score. The constructed fall dictionary is shown in Table 1.

TABLE 1
Fall Dictionary (MFS Dictionary)
Fall Dictionary (MFS Dictionary)

| Fall Word | Fall Score | Fall Word | Fall Score |
|---|---|---|---|
| Seizures | 25 | Crutches | 15 |
| Cane | 15 | Falling | 25 |
| Walkers | 15 | Already fallen | 25 |
| Intravenous appliances | 20 | Forgotten restrictions | 15 |
| Gait weakness | 10 | Intravenous devices | 20 |
| Gait disorders | 20 | Wheelchair | 0 |
| Diagnosis | 15 | Be verb | 0 |

The following describes the fall dictionary scores for the two different calculation schemes:
when negation before fall word is ignored, the process of calculating the fall dictionary score consists of:
Step 1: importing patient life;
Step 2: using Jieba word segmentation tool to obtain vector phrases;
Step 3: obtaining words about fall based on MFS dictionary;
Step 4: iterating through the obtained fall words;
Step 5: the score is the sum of the fall word scores;
when there is a negation, the score is multiplied by −1, the process of calculating the fall dictionary score includes:
Step 1: importing patient life;
Step 2: using Jieba word segmentation tool to obtain vector phrases;
Step 3: obtaining words about fall based on MFS dictionary;
Step 4: iterating through the obtained fall words;
Step 5: the score is the sum of the scores for fall words only and the opposite of the scores for fall words with negation.

TABLE 2
Comparison of fall scores of elderly in different
situations (with 8 electronic nursing files)

| | Fall scores that | Fall scores without |
|---|---|---|
| ID | ignore negations | ignoring negations |
| Narrative 1 | 15 | 15 |
| Narrative 2 | 15 | 15 |
| Narrative 3 | 0 | 0 |
| Narrative 4 | 0 | 0 |
| Narrative 5 | 25 | −25 |
| Narrative 6 | 55 | 55 |
| Narrative 7 | 25 | −25 |
| Narrative 8 | 30 | 30 |

As shown in Table 2, the fall scores of the 8 elderlies have been analyzed specifically. When the negation is ignored, the scores don't have negative cases and the lowest score is 0. The fall scores in Narrative 3 and Narrative 4 are 0, indicating that these two elderlies are currently in good health and have a low fall risk. These two elderlies need to maintain their current physical condition and maintain their daily habits. The low fall scores of the four elderlies in Narrative 1, Narrative 2, Narrative 5 and Narrative 7 indicate that the four elderlies are currently in a relatively stable physical condition with a certain fall risk, and they need to improve their current physical condition and enhance their quality of life to avoid falling. The high fall scores of the two elderlies in Narrative 6 and Narrative 8 indicate that the two elderlies are currently in poor physical condition and have a high fall risk. The two elderlies need to improve their current poor physical condition as soon as possible, and their family member or caregiver should be arranged to take care of them to prevent fall accidents.

As shown in Table 2, it can be seen that when the negation is included, the final fall word scores have negative cases and the lowest score is less than 0. The fall scores of the two elderlies in Narrative 3 and Narrative 4 are the same as the previous calculation, indicating that the two elderlies are in good health and have a low fall risk. The two elderlies need to maintain their current health and daily living habits; the fall scores of the two elderlies in Narrative 1 and Narrative 2 are the same as the previous calculation with the low fall scores, indicating that these four elderlies are in a stable health condition and have a certain fall risk. The fall scores of the two elderlies in Narrative 6 and Narrative 8 are 55 and 30 respectively, which means that the two elderlies are in poor physical condition and have a high fall risk, and they need to improve their poor physical condition as soon as possible. In Narrative 5 and Narrative 7, compared with the previous calculation, the fall scores both changed to −25, which means these two elderlies have low fall risk, so they should maintain their current physical condition and maintain their daily living habits.

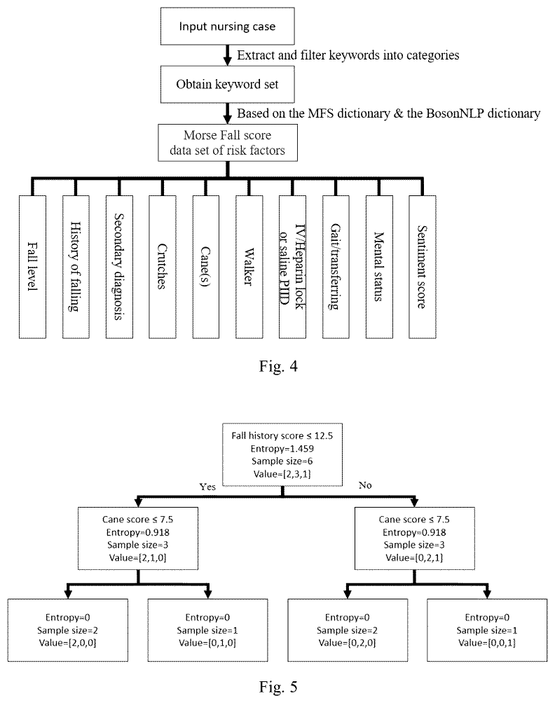

As shown in FIG. 4, steps for determining the variable set includes:
Step 1: extracting important keywords from patient's nursing records; the keywords include information closely related to the elderly such as physical condition, living condition, mental condition, medical condition and disease history;
Step 2: filtering the keywords and then getting them summarized and categorized;
Step 3: matching the summarized and categorized keywords with the MFS dictionary and BosonNLP dictionary to obtain the final variable set.

The extracted text features were analyzed by the MFS dictionary to obtain the results of the data set of risk factors as shown in Table 3.

TABLE 3
Text feature parsing table based on MFS dictionary

| Matter | Reference value | Score |
|---|---|---|
| History of fall | No | 0 |
| | Yes | 25 |
| Secondary diagnosis | No | 0 |
| (More than one diagnosis) | Yes | 15 |
| Ambulatory aid | A:None, on bedrest, uses W/C, or | 0 |
| | nurse assists | |
| | B:Crutches, cane(s), walker | 15 |
| | C:Furniture | 30 |
| IV/Heparin lock or saline | 0 | |
| PHD | Yes | 20 |
| Gait/transferring | A:Normal, on bedrest, immobile | 0 |
| | B: Weak (Uses touch for balance) | 10 |
| | C:Impaired (Unsteady, difficulty | 20 |
| | rising to stand) | |
| Mental status | A: Oriented to own ability | 0 |
| | B: Forgets limitation | 15 |

As shown in Table 4, data in the data set of risk factors includes fall level, history of falling, secondary diagnosis, crutches, cane, walker, intravenous appliances/heparin lock or saline PIID, gait/mobility, mental status, sentiment score, and Morse Fall Scale.

TABLE 4
Morse Fall Scale data set of risk factors

| Variable | Feature | Value type | Values range |
|---|---|---|---|
| X1 | Fall level | Integer | 1, 2, 3 |
| X2 | History of falling | Integer | 0, 25 |
| X3 | Secondary diagnosis | Integer | 0, 15 |
| X4 | Crutches | Integer | 0, 15 |
| X5 | Cane(s) | Integer | 0, 15 |
| X6 | Walker | Integer | 0, 15 |
| X7 | IV/Heparin lock or saline PIID | Integer | 0, 20 |
| X8 | Gait/transferring | Integer | 0, 10, 20, 30 |
| X9 | Mental status | Integer | 0, 15 |
| X10 | Sentiment score | Integer | (−∞, +∞) |
| Y | Morse Fall Scale | Integer | [0, +∞) |

Electronic nursing conversation data may be in the form of nursing record text Text (e.g. doctor's prescription to patient), conversation text of patient nursing process, etc. which needs to use methods such as text mining to process the feature quantity in the data such as extraction, fuzzy recognition and transformation, and finally form D′ₛ(x,T, y). This process can be noted as FS:

$${D}_{s}^{′} ( x , T , y ) = FS ( S ) = FS \{ LDA ( Text ) , LDA ( SR ( audio ) ) , … \}$$

wherein, LDA(Text) denotes text mining algorithm for dialogues, LDA(SR(audio)) denotes audio recognition into text. FS(S) in general embodies the techniques of pre-processing, pattern recognition, sentiment mining and feature extraction for transforming unstructured data of diverse dialogues into structured data.

The Morse Fall Scale decision-making table is constructed as shown in Table 5.

TABLE 5
Decision-making table based on Morse Fall Scale

| | Independent variable x | | | | | | | | | | Dependent |
|---|---|---|---|---|---|---|---|---|---|---|---|
| | | | | | | | IV/Heparin | | | | variable y |
| | | History | | | | | lock or | | | | Morse |
| Sample | Fall | of | Secondary | | | | saline | Gait/ | Mental | Sentiment | Fall |
| Cases | level | falling | diagnosis | Crutches | Cane(s) | Walker | PIID | transferring | status | score | scale |
| Narrative 1 | 1 | 0 | 0 | 0 | 0 | 15 | 0 | 0 | 0 | 1 | 15 |
| Narrative 2 | 1 | 0 | 0 | 0 | 0 | 15 | 0 | 0 | 0 | 12 | 15 |
| Narrative 3 | 1 | 0 | 0 | 0 | 0 | 0 | 0 | 0 | 0 | 12 | 0 |
| Narrative 4 | 1 | 0 | 0 | 0 | 0 | 0 | 0 | 0 | 0 | 10 | 0 |
| Narrative 5 | 2 | 25 | 0 | 0 | 0 | 0 | 0 | 0 | 0 | −9 | 25 |
| Narrative 6 | 3 | 25 | 0 | 0 | 15 | 15 | 0 | 0 | 0 | 5 | 55 |
| Narrative 7 | 2 | 25 | 0 | 0 | 0 | 0 | 0 | 0 | 0 | 11 | 25 |
| Narrative 8 | 2 | 0 | 0 | 0 | 15 | 15 | 0 | 0 | 0 | 7 | 30 |

In Table 5, the elderlies in Narrative 2, Narrative 3, Narrative 4 and Narrative 7 are in a good mood, positive and satisfied with their physical condition and the situation they are in, so they need to keep their good state of mind to maintain a low fall risk; the elderlies in Narrative 1, Narrative 6 and Narrative 8 are in a stable mood, smooth and normal, able to accept their physical condition and the environment they are in, these elderlies need to maintain or slightly improve their mood to reduce the fall risk; the elderly in Narrative 5 is obviously depressed and negative, dissatisfied with his health condition and the environment he is in. This elderly should adjust mood in time, and his caregiver or relatives should give him necessary help to come out of negative mood and positively face his current situation for health and reducing the probability of fall.

Decision Tree is a decision-making analysis method that evaluates the project risk and judges its feasibility by forming a decision tree to find the probability that the expected value is greater than or equal to zero on the basis of the known probability of occurrence of various situations, and is a graphical method that intuitively uses probability analysis.

Since this kind of decision-making branches is drawn like the branches of a tree, it is called a decision tree. In machine learning, a decision tree is a predictive model that represents a mapping relationship between object attributes and object values.

Entropy indicates the clutter degree of a system and is used in generating tree algorithm using algorithms ID3, C4.5 and C5.0. Entropy refers to a measure of the state of some material system, the degree to which the state of some material system may occur, the essence of entropy is the “intrinsic degree of disorder” of a system, that is:

$$S ( {p}_{1} , {p}_{2} , … , {p}_{n} ) = - K {\sum }_{i = 1}^{n} {p}_{i} {log}_{2} {p}_{i}$$

wherein, i marks all possible samples in the probability space, pᵢ denotes the chance of occurrence of that sample, and K is an arbitrary constant associated with the unit selection. This metric is based on the concept of entropy in informatics theory. A decision tree is a tree structure in which each internal node represents a test on an attribute, each branch represents a test output, and each leaf node represents a category. Classification tree (decision-making tree) is a very common classification method. It is a kind of supervised learning. A bunch of samples are given that each sample has a set of attributes and a category which are determined in advance. A classifier is obtained by learning, which is able to give the correct classification to the emerging objects. Such a machine learning is called supervised learning.

In Table 5, the fall scores of Method 1 are taken and fall risks of the elderlies are graded according to the fall criteria with a total of three levels, as well as the specific composition of the Morse scores which include fall risk level, history of falling, secondary diagnosis, crutches, cane(s), walker, IV/Heparin lock or saline PIID, gait/transferring, mental status, and sentiment score.

As shown in FIG. 5, the decision tree finally selected six cases out of eight as the test set, respectively two cases with fall risk level 1, three cases with fall risk level 2, and one case with fall risk level 3. when the fall history score is less than or equal to 12.5, three cases are classified: two cases with a fall risk level 1 and one case with a fall risk level 2; conversely, when the fall history score is greater than 12.5, three cases are classified: two cases with a fall risk level 2 and one case with a fall risk level 3. In the cases with a fall history score less than or equal to 12.5, there are two cases with a cane score less than or equal to 7.5, which are two cases with a fall risk level 1;conversely, in the cases with a fall history score less than or equal to 12.5, there is one case with a cane score greater than 7.5, which is one case with a fall risk level 2. Among the cases with a fall history score greater than 12.5, there are two cases with a cane score less than or equal to 7.5, which are two cases with a fall risk level 2. Among the cases with a fall history score greater than 12.5, there is one case with a cane score greater than 7.5, which is one case with a fall risk level 3.

A systematic clustering method is used to perform unsupervised learning of electronic nursing file data. The process enables the partitioning of these electronic nursing files by the number of clusters required by the user. This approach is independent of the category variables in the data set and thus more flexible than decision tree segmentation. The results of this learning approach enable stratified health management of relevant patients. The clustering table is derived from a systematic cluster analysis, which lists the stepwise clustering of variables, wherein the clustering method is intergroup linkage, and the measurement interval is the squared Euclidean distance. The first column indicates which step of clustering it is; the second and third columns indicate which samples or subclasses are clustered together in this step (the subclasses clustered together in the previous step will be named after the previous one for that subclass); the fourth column coefficients indicate the distance between the individuals or subclasses of the samples clustered in this step; the fifth and sixth columns indicate which step the subclasses generated in will be clustered with the samples in the previous step in this step; the seventh column, the next stage, indicates which step the subclasses generated in this step will be used.

TABLE 6
Centralized schedule using average linkage (between groups)

| | Combinatorial clustering | | | The step clustering first appears | | Next |
|---|---|---|---|---|---|---|
| Step | Clustering 1 | Clustering 2 | coefficient | Clustering 1 | Clustering 2 | step |
| 1 | 5 | 7 | .000 | 0 | 0 | 6 |
| 2 | 3 | 4 | .000 | 0 | 0 | 5 |
| 3 | 1 | 2 | .000 | 0 | 0 | 4 |
| 4 | 1 | 8 | 300.667 | 3 | 0 | 5 |
| 5 | 1 | 3 | 1080.800 | 4 | 2 | 7 |
| 6 | 5 | 6 | 1981.467 | 1 | 0 | 7 |
| 7 | 1 | 5 | 4185.125 | 5 | 6 | 0 |

In Table 6, the elderlies in cases 1 to 8 are marked as 1 to 8. The clustering group table above presents the process of variables being aggregated up step by step: the first row is 5 and 7, i.e., case 5 and case 8 are aggregated first with a distance coefficient of 0, which is the smallest; similarly, the distance coefficients of case 3 and case 4, case 1 and case 2 are all 0, so they are each classified into one category. In the fourth row, case 1 and case 8 are aggregated. The other rows are explained in the same way, i.e., the smaller the distance coefficient is, the more they are aggregated first.

TABLE 7
Cluster membership table using average linkage (between groups)

| Case | 4 clusters | 3 clusters | 2 clusters |
|---|---|---|---|
| 1: Narrative1 | 1 | 1 | 1 |
| 2: Narrative2 | 1 | 1 | 1 |
| 3: Narrative3 | 2 | 1 | 1 |
| 4: Narrative4 | 2 | 1 | 1 |
| 5: Narrative5 | 3 | 2 | 2 |
| 6: Narrative6 | 4 | 3 | 2 |
| 7: Narrative7 | 3 | 2 | 2 |
| 8: Narrative8 | 1 | 1 | 1 |

Table 7 shows the cluster membership table, when the number of clusters is four, case 1, case 2 and case 8 are the first category, case 3 and case 4 are the second category, case 5 and case 7 are the third category and case 6 is the fourth category; when the number of clusters is three, case 1, case 2, case 3, case 4 and case 8 are the first category, case 5 and case 7 are the second category, and case 6 is the third category; when the number of clusters is two, case 1, case 2, case 3, case 4 and case 8 are the first category, and case 5, case 6 and case 7 are the third category.

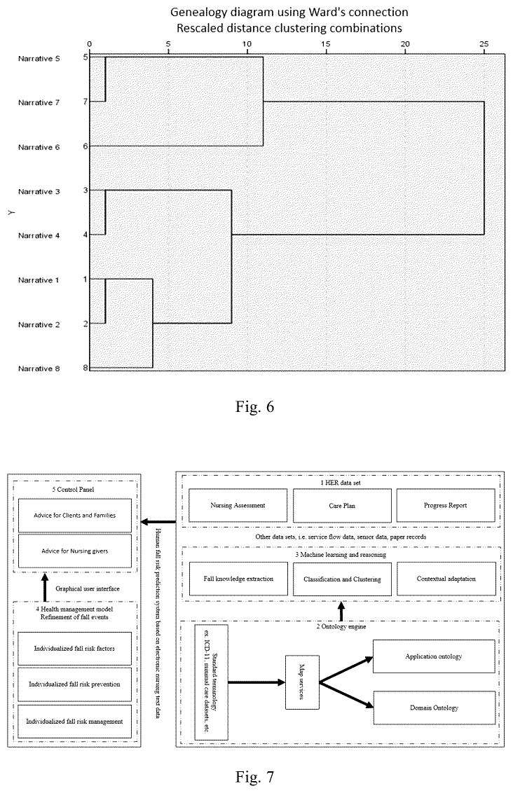

As shown in FIG. 6, cases are classified. Starting from the outermost line, for example, if the variables are divided into two categories, Case 5, Case 6, and Case 7 are divided into one category, and the other cases are divided into one category; if they need to be divided into three categories, they are divided from the second level, and Case 6 is divided into one category, Case 5 and Case 7 are divided into one category, and the other cases are divided into one category; if they need to be divided into four categories, they are divided from the third level, and Case 5 and Case 7 are divided into one category, and case 6 into one category, case 3 and case 4 into one category, and other cases into one category.

A system for predicting human fall risk based on ENT data, as shown in FIG. 7, which is for performing the method for predicting human fall risk based on ENT data as described above, wherein the system comprises: a data acquisition module, a data pre-processing module, a text feature extraction module, a MFS dictionary module, an iterative risk prediction module, a fall event prevention and control module, and a feedback module;
the data acquisition module is used for acquiring the user's electronic unrsing text data and inputting the data into the data pre-processing module; the ENT data including nursing assessments, nursing plans, progress reports and other data sets; other data sets include service flow data, sensor data, and paper records;
the data pre-processing module is used for pre-processing the ENT data, wherein the pre-processing comprises filtering the ENT data for corresponding features, removing duplicate features, and completing missing features;
the text feature extraction module is used for extracting features from the ENT data processed by the data pre-processing module;
the MFS dictionary module is used for analyzing the extracted text features to obtain the data set of risk factors; that is, parsing the extracted text features includes constructing ontology engine, creating map services with standard terms such as ICD-11, minimum nursing data set, and applying them to application ontologies and domain ontologies;
the iterative risk prediction module is used for selecting features in the data set of risk factors using a decision tree algorithm to obtain prediction result of patient's fall risk, and inputting the prediction result into the fall event prevention and control module;
the fall event prevention and control module is used for constructing fall risk prevention strategy based on the prediction result; the strategy includes individualized fall risk factors, individualized fall risk prevention, and individualized fall risk management;
the feedback module is used to feedback the fall risk prevention strategy generated by the fall event prevention and control module to the user.

The system of the present invention is implemented in the same embodiment as the method.

The above descriptions are only examples of the invention, and are not used to limit the protection scope of the invention. For those skilled in the art, the application can have various modifications and changes. Any modification, equivalent replacement and improvement. made within the core content and principle of this invention shall be included in the protection scope of this invention.

1. A method for predicting human fall risk based on electronic nursing text (ENT) data, characterized in that the method comprises the following steps: obtaining an ENT data set, pre-processing data in the ENT data set, and constructing a Morse Fall Scale (MFS) dictionary with the pre-processed data; extracting features from the ENT data of patients to be predicted with a natural language processing technology; analyzing the extracted text features by the MFS dictionary to obtain a data set of risk factors; training a decision tree algorithm by using the data set of risk factors to obtain a prediction result of the patient's fall risk; clustering and precisely nursing the patients according to the prediction result.

2. The method for predicting human fall risk based on ENT data according to claim 1, wherein the process of constructing the MFS dictionary comprises: performing sentiment score mining and fall dictionary score mining on all ENT data in the ENT data set; constructing the MFS dictionary based on the results of sentiment score mining and fall dictionary score mining.

3. The method for predicting human fall risk based on ENT data according to claim 2, wherein the process of performing sentiment score mining on the ENT data comprises: using Jieba word segmentation tool to the ENT data to obtain vector phrases; using natural language processing technology to extract sentiment words of the vector phrases; classifying all sentiment words into sentiment words with negation, sentiment words without negation, and other sentiment words; respectively applying sentiment word score mechanisms of sentiment words with negation, sentiment words without negation, and other sentiment words to compute sentiment scores of sentiment words with negation, sentiment words without negation, and other sentiment words; summing all the sentiment scores to get total score for the sentiment words.

4. The method for predicting human fall risk based on ENT data according to claim 3, wherein the process of applying sentiment word score mechanism of sentiment words with negation to calculate sentiment score of sentiment words with negation includes:
Step 1: segmenting the document into words to find the sentiment words, negation words and adverbs of degree;
Step 2: determining whether each sentiment word is preceded by negation word and adverb of degree, and combining the negation word and the adverb of degree into a group;
Step 3: computing score of the sentiment words with negation and weight of the adverb of degree according to the NLP dictionary; multiplying sentiment weight of the sentiment words by −1 if there is a negation; multiplying degree value of the adverb of degree if there is an adverb of degree;
Step 4: taking opposite number of initial score and then multiplying it with the weight of the adverb of degree to get the sentiment score of the sentiment words with negations; summing up the sentiment scores of sentiment words; those greater than 0 are classified as positive, and those less than 0 are classified as negative, wherein the absolute magnitude of the sentiment score reflects the degree of positivity or negativity.

5. The method for predicting human fall risk based on ENT data according to claim 4, wherein the weight of the adverb of degree is calculated by formula:
$$Score ( w ) = {log}_{2} \frac{freq ( w , positive ) * freq ( negative )}{freq ( w , negative ) * freq ( positive )}$$
wherein, freq (w, positive) is the number of occurrences of a word w in positive texts, freq (positive) is the total number of each word in each nursing text, freq (negative) is the total number of negative words in each nursing text, and freq (w, negative) is the number of occurrences of a word w in negative text.

6. The method for predicting human fall risk based on ENT data according to claim 3, wherein the process of applying sentiment word score mechanism of sentiment words without negation to calculate sentiment score of sentiment words without negation includes: calculating initial score of sentiment words without negation and degree adverb weight; multiplying the initial score with the degree adverb weight to obtain the sentiment score of the sentiment words without negation.

7. The method for predicting human fall risk based on ENT data according to claim 2, wherein the process of performing fall dictionary score mining on the ENT data includes: constructing fall dictionary; using Jieba word segmentation tool to the ENT data to obtain vector phrases; using the fall dictionary to extract fall words in the vector phrases; calculating score of each fall word, and summing all the scores to obtain fall dictionary score.

8. The method for predicting human fall risk based on ENT data according to claim 1, wherein data in the data set of risk factors includes fall level, fall history, secondary diagnosis result, crutch, cane, walker, intravenous appliances/heparin lock or saline indicators, gait/mobility, mental status, sentiment score and the Morse Fall Scale.

9. The method for predicting human fall risk based on ENT data according to claim 1, wherein the process of processing the data in the data set of risk factors using the decision tree algorithm comprises:
Step 1: constructing decision tree, taking the Morse Fall Scale in the data set of risk factors as root node of the decision tree, and classifying patients according to the root node:
Step 2: querying each subclass to determine whether the classification result of each subclass is correct; if yes, taking branch end node as leaf node of the decision tree;
otherwise, selecting a non-parent node attribute and repeating the Step 1;
Step 3: selecting a non-parent node's attribute, and continuing to classify results in the Step 1 according to attribute score; classification result is final prediction result.

10. A system for predicting human fall risk based on ENT data, which is for performing the method for predicting human fall risk based on ENT data as claimed in any one of claims 1 to 9, wherein the system comprises: a data acquisition module, a data pre-processing module, a text feature extraction module, a MFS dictionary module, an iterative risk prediction module, a fall event prevention and control module, and a feedback module;
the data acquisition module is used for acquiring the patient's ENT data and inputting the data into the data pre-processing module;
the data pre-processing module is used for pre-processing the ENT data, wherein the pre-processing comprises filtering the ENT data for corresponding features, removing duplicate features, and completing missing features;
the text feature extraction module is used for performing text feature extraction on the data processed by the data pre-processing module;
the MFS dictionary module is used for analyzing the extracted text features to obtain the data set of risk factors;
the iterative risk prediction module is used for selecting features in the data set of risk factors using a decision tree algorithm to obtain prediction result of human fall risk, and inputting the prediction result into the fall event prevention and control module;
the fall event prevention and control module is used for constructing fall risk prevention strategy based on the prediction result;
the feedback module is used to feedback the fall risk prevention strategy generated by the fall event prevention and control module to the patient.
